(19) United States
(12) Patent Application Publication
(10) Pub. No.: US 2012/0055471 A1
(43) Pub. Date: Mar. 8, 2012

(54) VALVE ACCESSORY FOR AIDING SPEECH DURING NON-INVASIVE RESPIRATORY THERAPY

(75) Inventors: Noam Hadas, Tel-Aviv (IL); Jacob Zomer, Nes Tziona (IL)

(73) Assignee: SLP Ltd., Tel Aviv (IL)

(21) Appl. No.: 12/876,139

(22) Filed: Sep. 5, 2010

(51) Int. Cl. A61M 16/06; A62B 18/08; A61M 16/00

(52) U.S. Cl. 128/201.19; 128/205.24; 128/207.18

(57) ABSTRACT

A valve arrangement interposed between a compressor and a user, in a respiratory pressure support system. A connecting element connects a breathing tube to a human interface apparatus. A shutter is formed within the connecting element. The shutter is placed in a first position allowing pressurized air to flow from the compressor to the user. This shutter is placed in a second position preventing pressurized air from flowing from the compressor to the user.

### FIELD AND BACKGROUND OF THE INVENTION

The present invention relates to a respiratory therapy accessory and, more particularly, to a valve for Continuous Positive Airway Pressure (CPAP) masks and non-invasive ventilation devices to allow free oral expression such as speech during therapy.

Sleep apnea is a sleep disorder characterized by having multiple pauses in breathing or shallow breaths during sleep. Each pause in breathing, called an apnea, can last from 10 seconds to minutes, and may occur 50 times or more an hour.

For moderate to severe sleep apnea, the most common treatment is the use of a continuous positive airway pressure (CPAP) device, which ensures continuous ventilation by keeping the upper airways open during sleep by means of a flow of pressurized air. The patient typically wears a plastic mask (nasal, oral, nasal-oral or facial), which is connected by a flexible tube to a small bedside CPAP machine. The CPAP machine generates the required air pressure to keep the patient airways open during sleep. Advanced models may warm and/or humidify the air and monitor the patient's breathing to insure proper treatment. The CPAP machine provides a constant pressure to the patient, whereas a Variable Positive Airway Pressure (VPAP) machine continuously adjusts the pressure according to the patient's needs, and a Bilevel/BiPAP machine lowers the pressure during exhale to allow the patient less work during exhale, and is used in more severe cases.

Although CPAP therapy is extremely effective in reducing apneas, most patients find it extremely uncomfortable. Many patients refuse to continue the therapy or fail to use their CPAP machines on a nightly basis. One cause of discomfort is the difficulty (near inability) of conversing or even yawning during therapy due to the pressurized air entering the mouth through the nose. Currently, in order to answer the phone or converse with another, it is necessary to remove the entire contraption from face (and head), only to be replaced and repositioned at a later stage. It is important to note that replacing the mask is a difficult and lengthy process since it is important to prevent any leaks from the mask, so precise fitting and placement is required.

It would be highly advantageous to have a mechanism or accessory which allows for the temporary cessation of pressurized air flow to the patient without necessitating either removal of the mask or powering down of the machine.

While reference is made regularly herein to a CPAP system, the current innovation can be equally applied to any respiratory pressure support system and/or non-invasive ventilation device suffering from the same or similar drawbacks as detailed above.

Typically therapeutic non-invasive ventilation systems use tubing with an inner lumen having a 22 mm diameter. Whilst reference is made to apparatuses compatible with the 22 mm size tubing, it is hereby made clear that the current innovation can be equally applied to tubing that have a bore range between 8 mm and 35 mm, including at least 8.5 mm tubing, 15 mm tubing and 30 mm tubing according to at least the specifications detailed in ISO (International Organization for Standardization) 5356-1.

### TERMS AND DEFINITIONS

For the sake of clarity and continuity, definitions and terminology relating to the breathing apparatuses will substantially follow the terms and definitions detailed in ISO 17510-2 and ISO 5356-1 partially copied here. Copies of ISO standards 17510-2 and 5356-1 are available for purchase from the International Organization for Standardization, 1, ch. de la Voie-Creuse, CP 56, 1211 Genève 20, Switzerland.

Anti-asphyxia valve—valve used on a naso-oral mask, which is open to atmosphere when the sleep apnea breathing therapy equipment is not providing adequate pressure at the mask (i.e. less than 3 cm H₂O) and that is closed to atmosphere when the sleep apnea breathing therapy equipment is providing adequate pressure at the mask.

Exhaust flow—flow from the mask or application accessories to atmosphere other than the leak due to improper seal to the face.

NOTE 1 The exhaust flow can pass through [dedicated] openings in the mask, [in] the connecting element and the mask, or through the anti-asphyxia valve.

NOTE 2 The exhaust flow discharges exhaled gases to atmosphere to reduce rebreathing of CO₂.

Headgear—part that is used to fix the mask to the patient.

Mask—part which provides the interface between the patient and the patient connection port.

NOTE According to their application, masks are divided into: nasal masks, oral masks or nasal-oral masks.

Patient connection port—port where the breathing gas pathway connects to the mask.

The terms ‘patient’ and ‘user’ are used interchangeably in the scope of this document.

In some embodiments of the current invention, the innovative valve is housed in a conical connector interposed between the breathing tube connected to the compressor and the patient connection port. In other embodiments, the innovative valve can be incorporated into the breathing tube itself, the patient connection port, any other accessory connected between the breathing tube and the patient connection port such as a swivel connector, or in the mask itself.

### SUMMARY OF THE INVENTION

The current innovation allows a user to speak while wearing the mask, something which is currently difficult to do due to the pressurized air entering the mouth through the nose. The innovation is a valve that fits between the mask and tube leading to the compressor. In the first, ‘open’ position the valve allows air to flow from the compressor to the mask normally. In the second, ‘closed’, position the shutter is depressed/actuated, thereby blocking off the airflow from the compressor and allowing air from the surrounding environment to enter the mask. This allows the user to speak or yawn without difficulty or the need to remove the mask. The valve shutter is returned to the ‘open’ position by force of the air pressure, when no longer depressed/actuated.

According to the present invention there is provided a valve arrangement for interposing between a compressor and a user, in a respiratory pressure support system, including: (a) a shutter operative to be placed alternately in a first position for allowing positive pressurized air flow from the compressor to the user and in a second position for preventing positive pressurized air flow from the compressor to the user, wherein the shutter is reversibly movable between the first and second positions.

According to further features in preferred embodiments of the invention the shutter includes a handle for reversibly moving the shutter from the first position to said second position and wherein the handle is shaped ergonomically for uninhibited manual manipulation of the shutter.

According to still further features in the described preferred embodiments where the valve arrangement further includes a biasing element, such as a torsion spring, for biasing the shutter in the second position in the substantial absence of pressurized air flow.

According to further features in preferred embodiments of the invention, the shutter is held in the first position by positive air pressure flowing from the compressor to the user.

According to further features in preferred embodiments of the invention the valve is incorporated in an element selected from the group including: (i) a breathing tube that is operative to be connected to the compressor; (ii) a connecting element with conical connectors for connecting between the breathing tube and a human interface apparatus that is operative to be affixed to the user; and (iii) the human interface apparatus which is selected from the group including: (A) a nasal mask; (B) an oral mask; and (C) a nasal-oral mask.

According to further features in preferred embodiments of the invention the valve arrangement further including: (b) an actuator for electrically moving the shutter reversibly between the first and second positions.

According to still further features, the actuator is operationally coupled to an element selected from the group including: (i) a breathing tube that is operative to be connected to the compressor; (ii) a connecting element with conical connectors for connecting between the breathing tube and a human interface apparatus that is operative to be affixed to the user; and (iii) said human interface apparatus which is selected from the group including: (A) a nasal mask; (B) an oral mask; and (C) a nasal-oral mask.

According to still further features the actuating element is further configured to power-down the compressor when actuated.

According to further features the valve arrangement further includes a microphone for detecting speech of the user, wherein, the actuator is operative to move the shutter to the second position in response to detection of speech by the microphone.

According to further features in preferred embodiments of the invention wherein, when the shutter is in the second position, the valve is configured, to allow free communication of ambient air between the valve and the user.

According to another embodiment a valve arrangement, operative to be interposed between a compressor and a user, in a respiratory pressure support system including: (a) a connecting element for operatively connecting a breathing tube and a human interface apparatus; and (b) a shutter formed within the connecting element, the shutter being operative to be placed alternatively in a first position for allowing positive pressurized air flow from the compressor to the user and in a second position for preventing positive pressurized air flow from the compressor to the user, wherein the shutter is reversibly movable between the first position and the second position.

According to further features wherein the connecting element includes conical shaped connecting edges for reversibly operatively connecting the connecting element to the compressor and to the human interface apparatus.

According to still further features wherein the connecting element is a connector having a diameter ranging in length between 8 mm and 35 mm.

According to still further features wherein the shutter in the first position defines an air vent between the shutter and the connecting element.

According to further features wherein the human interface apparatus is selected from the group including: (i) a nasal mask; (ii) an oral mask; and (iii) a nasal-oral mask.

### BRIEF DESCRIPTION OF THE DRAWINGS

Various embodiments are herein described, by way of example only, with reference to the accompanying drawings, wherein:

FIG. 1a is an isometric side view of an embodiment of the invention in an open position;

FIG. 1b is an isometric side view of an embodiment of the invention in a closed position;

FIG. 1c is an isometric front view of an embodiment of the invention in an open position;

FIG. 1d is an isometric front view of an embodiment of the invention in an closed position;

FIG. 2 is a side view of an embodiment of the invention in a partially open position;

FIG. 3 is a front view of an embodiment of the invention;

FIG. 4 is a top view of an embodiment of the invention in a partially open position;

FIG. 5 is an illustrative depiction of a nasal mask on a user;

FIG. 6 is an illustrative depiction of a nasal-oral mask on a user;

FIG. 7a is an illustrative depiction of an embodiment of the invention interposed between a breathing tube and nasal mask;

FIG. 7b is a magnified view of an embodiment of the current invention;

FIG. 8 is a schematic diagram of a typical non-invasive ventilation system.

### DESCRIPTION OF THE PREFERRED EMBODIMENTS

The principles and operation of a speaking valve for a non-invasive ventilation apparatus according to the present invention may be better understood with reference to the drawings and the accompanying description.

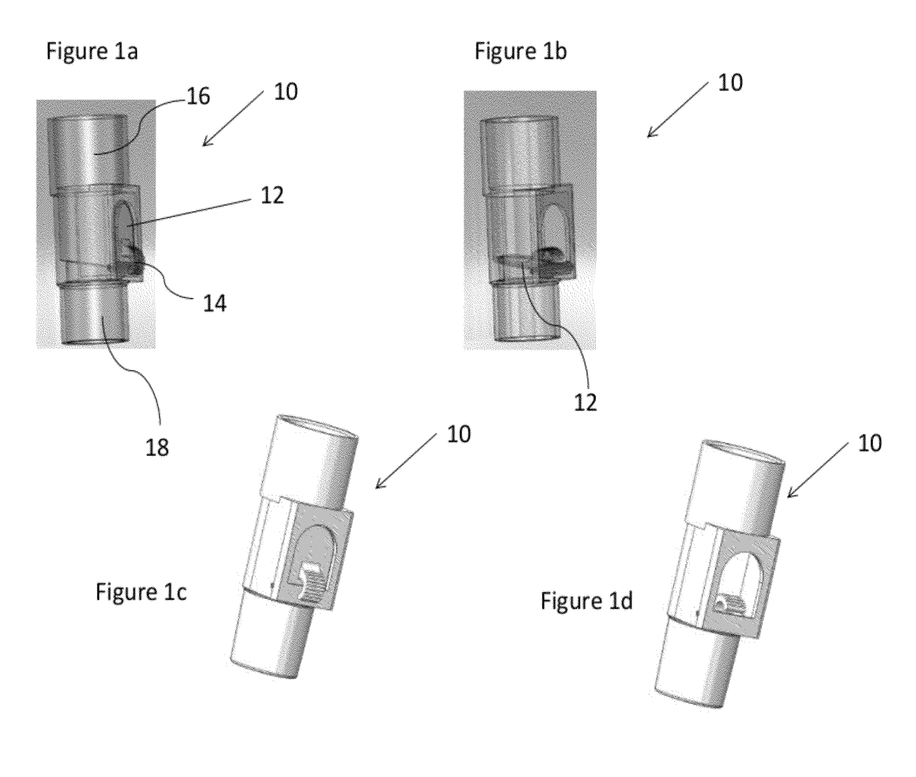

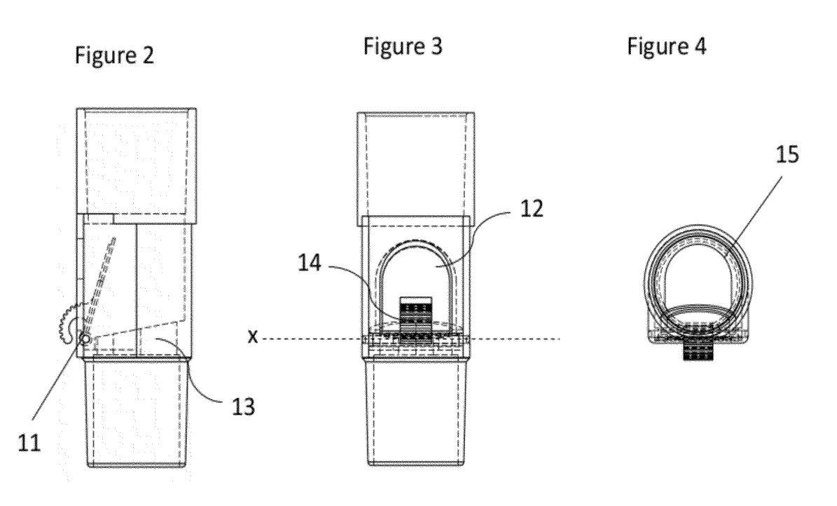

Referring now to the drawings, FIGS. 1-4 illustrate the innovative valve in various perspective views. FIG. 2 is a side view of an embodiment of valve 10 where the shutter is partly actuated. FIG. 3 is front view of an embodiment of the innovative valve. FIG. 3 is a top view of the valve. In the current embodiment, the valve is housed in a connecting piece having conically shaped edges in accordance with ISO 5356-1 “Anaesthetic and respiratory equipment—Conical connectors—Part 1: Cones and sockets”. The valve arrangement is intended to be attached on its distal end to a breathing tube connected to an air compressor and on its proximal end connected either directly or indirectly to a breathing mask.

The embodiment chiefly described and referred to herein is formed in a connecting piece conforming at least to the regulations set forth in both ISO 5356-1 and ISO 17510-2. It is to be understood that the current innovation can potentially be incorporated into existing components, including: breathing tube, connecting element and even the mask itself.

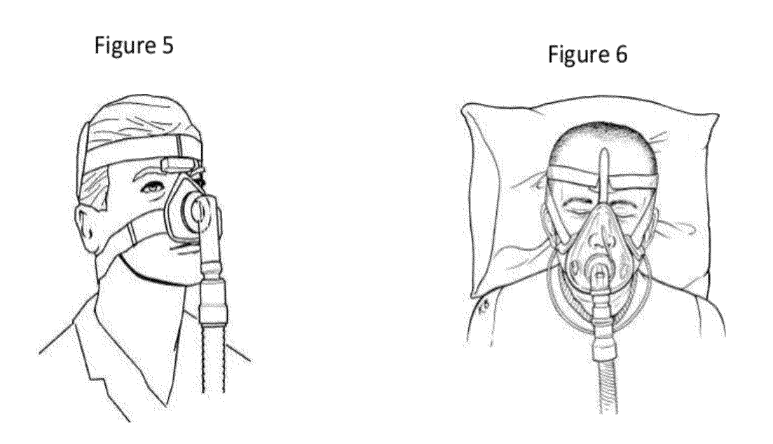

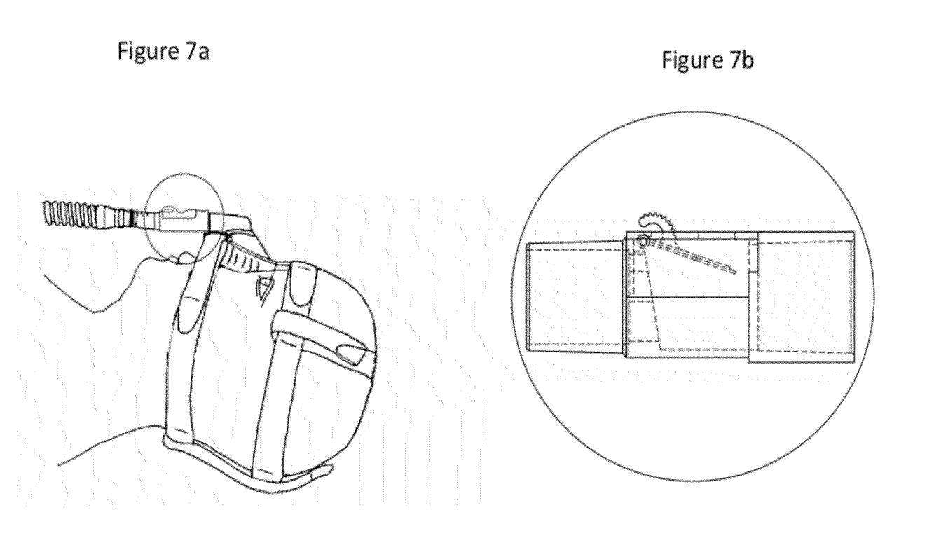

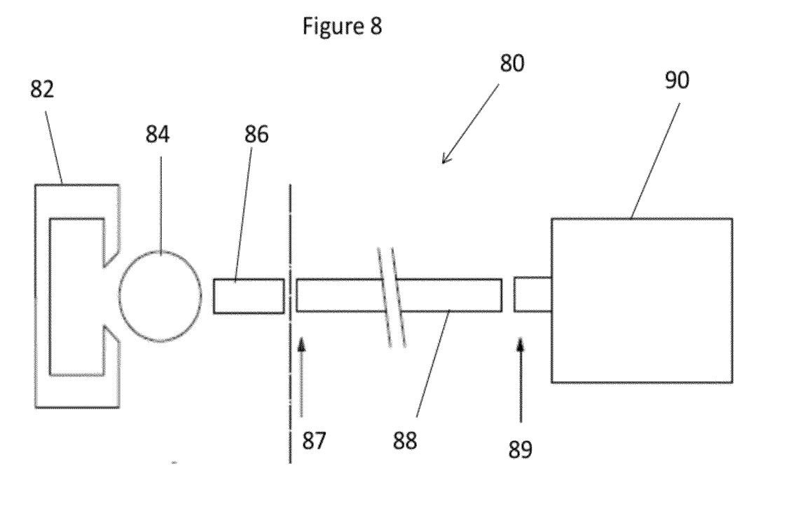

FIGS. 1a and 1c depict one embodiment of the current invention where the valve is in a position which allows the free flow of pressurized air from a compressor through a breathing tube, through the connecting piece in question and on to the mask. In order to provide a clear context, a schematic diagram of a typical non-invasive ventilation system 80 is provided in FIG. 8. Typically, a headgear arrangement 82 is provided for affixing a mask 84 to the face of a user. As mentioned previously, masks are typically configured for oral, nasal or oral-nasal use (FIGS. 5 and 6 depict exemplary configurations of nasal and oral-nasal masks respectively). An optional connecting element 86 is often found in apnea therapy ventilation systems, usually adapted to provide increased mobility to the user wearing a mask. The current innovative valve can be attached to, or in place of, the typical connecting element. Potentially, in order for the valve to be located in a comfortable position for use, a multiplicity of connecting elements, an elongated connecting element or an additional short tube can be attached to the mask and the innovative valve attached thereto, approximately at an elbow length from the face mask, or as deemed most comfortable for the user. Patient connection point 87 is the proximal port on breathing tube 88 to which is connected the human interface apparatus and/or application accessories provided uniquely for the user. The human interface apparatus is one of, but not limited to: an oral mask, a nasal mask and an oral-nasal mask. Gas output port connector 89 is the port connector to which breathing tube 88 is connected. In one embodiment of the invention, the gas output is a pressurized air. Air compressor 90 sends compressed air via the breathing tube, optional connecting elements and a human interface apparatus to the user. FIG. 7a is an illustrative depiction of an embodiment of the invention interposed between a breathing tube 88 and nasal mask 84. FIG. 7b is a magnified view of an embodiment of the current invention.

Referring now back to FIGS. 1-4, valve arrangement 10 is depicted in FIGS. 1a and 1c with shutter 12 in the ‘open’ position, allowing the flow of pressurized air to pass substantially unimpeded through the valve. The connecting element housing the innovative valve is constructed in accordance with at least the regulations set forth in ISO 17510-2 and, specifically, to minimize pressure drop when air flows through the housing. Proximal edge 16 is a female conical connecting edge, for coupling connecting element either directly or indirectly to a mask 84. Distal edge 18 is a male conical connecting edge for coupling the connecting element either directly or indirectly to breathing tube 88. Shutter 12 is reversibly actuated by manipulating ergonomically designed handle 14 (see most clearly in FIGS. 1c and 1d) to reversibly move the shutter from the first open position to the second, closed, position depicted in FIGS. 1b and 1d. Here, the term ‘ergonomically designed’ is used when referring to handle 14 which is specifically designed for use in innovative valve 10 and where, primarily, the design of the handle is formed in a ridged arc having a sufficient length so as to allow the user to comfortably and freely move the shutter from the first position to the second position and back using a single finger and without being impeded by the casing of the connector element, and without blocking the ambient air port on the housing with the finger. The ridged formation of the handle provides the necessary traction to both close the valve and open it, should the need arise.

The standard operation for actuating the shutter is to apply direct or slightly rotational force to handle 14, thereby forcing shutter 12 against the flow of positively pressurized air moving within the lumen in the distal-to-proximal direction. The shutter follows an arc about the x-axis (delineated in FIG. 3) of the shutter hinge 11 (FIG. 2) until making contact with a corresponding groove 15 (FIG. 4) within the internal lumen 13 of the connecting piece, substantially covering the entire circumference of the internal lumen and restricting, substantially entirely, the flow of pressurized air from the compressor, while at the same time allowing the free communication of ambient air (i.e. non-compressed air from the immediate environment external to system 80) between the user and the environment external to system 80. At this stage, a user is capable of natural sounding oral and nasal expression, unimpeded by the pressurized air or by a closed space formed by the mask volume. Potentially, valve 10 can be configured only to restrict the flow of pressured air. Such a configuration would be less practical for use with an oral or oral-nasal mask, but still applicable for use with a nasal mask.

Once pressure is reduced or removed from handle 14, the pressurized air forces the shutter to retreat in the reverse direction along the arc about the x-axis previously described and finally return to the first, open, position and remain there.

In a further potential embodiment, shutter 12, can be biased in the second position by a biasing element, such as a torsion spring (not shown), which urges the shutter to the second position in the absence of substantial air pressure. In this embodiment, valve 10 serves dually as a push-to-talk valve described above as well as an anti-asphyxia valve. In the latter function (anti-asphyxia valve), in the event of substantial or complete reduction in air pressure, shutter 12 is biased towards the second position by the biasing element, thereby introducing air from the external environment and providing an additional exhaust portal for expelled CO₂. ‘Substantial air pressure’ is hereby clarified as at the most 3 cm H₂O or less. The simplistic anti-asphyxia function of the innovative valve can prevent asphyxia resulting from re-breathing expelled CO₂ gases. Further potentially, in the absence of a biasing element, shutter 12 can be returned to the first position manually, by drawing handle 14 proximally towards the user, employing the same movement for actuating the device, only this time in reverse.

In other possible configurations, the functional elements of innovative valve 10 can be incorporated into other elements of system 80 including, but not limited to: breathing tube 88, connecting element 86 and mask 84. ‘Connecting elements’ referred to here and elsewhere in this document are generically used to refer to any element with appropriate conical connecting edges including but in no way limited to, locking connectors, swivel connectors, extension elements, etc. Conversely and/or additionally, functional elements of additional components can be incorporated into valve 10. In an exemplary, non-limiting embodiment, an aperture, defined between the upper ridge of shutter 12 and the corresponding section of the connector can perform the function of an exhaust port providing for exhaust flow as required by ISO 17510-2.

In a further embodiment of the current innovation, valve 10 can be additionally or alternatively actuated electronically. In some embodiments, an actuating element such as a button or switch can remotely automatically control valve 10. The button or switch can be situated anywhere practical on system 80 including, but not limited to: breathing tube 88, connecting element 86 and mask 84. In a further embodiment, the switch or button can be further configured to additionally power-down the compressor when actuated. In a still further embodiment, a microphone is incorporated into system 80, preferably positioned to near the mouth of the users or valve 10. The microphone is configured to be voice reactive or speech activated so that when the microphone detects speech from the user, valve 10 is automatically actuated so as to allow the user to speak freely as previously discussed.

While the invention has been described with respect to a limited number of embodiments, it will be appreciated that many variations, modifications and other applications of the invention may be made. Therefore, the claimed invention as recited in the claims that follow is not limited to the embodiments described herein.

What is claimed is:

1. A valve arrangement for interposing between a compressor and a user, in a respiratory pressure support system, comprising:
(a) a shutter operative to be placed alternately in a first position for allowing positive pressurized air flow from the compressor to the user and in a second position for preventing positive pressurized air flow from the compressor to the user, wherein said shutter is reversibly movable between said first position and said second position.

2. The valve arrangement of claim 1 wherein said shutter includes a handle for reversibly moving said shutter from said first position to said second position.

3. The valve arrangement of claim 2 wherein said handle is shaped ergonomically for uninhibited manual manipulation of said shutter.

4. The valve arrangement of claim 1 further comprising:
(b) a biasing element, said biasing element for biasing said shutter in said second position in the substantial absence of said pressurized air flow.

5. The valve arrangement of claim 4 wherein said biasing element is a torsion spring.

6. The valve arrangement of claim 1 wherein said shutter is held in said first position by positive air pressure flowing from the compressor to the user.

7. The valve arrangement of claim 1 wherein the valve is incorporated in an element selected from the group including:
(i) a breathing tube that is operative to be connected to the compressor;
(ii) a connecting element with conical connectors for connecting between said breathing tube and a human interface apparatus that is operative to be affixed to the user; and
(iii) said human interface apparatus.

8. The valve arrangement of claim 7 wherein said human interface apparatus is selected from the group including:
(A) a nasal mask;
(B) an oral mask; and
(C) a nasal-oral mask.

9. The valve arrangement of claim 1 further comprising:
(b) an actuator for electrically moving said shutter reversibly between said first and second positions.

10. The valve arrangement of claim 9 wherein said actuator is operationally coupled to an element selected from the group including:
(i) a breathing tube that is operative to be connected to the compressor;
(ii) a connecting element with conical connectors for connecting between said breathing tube and a human interface apparatus that is operative to be affixed to the user; and
(iii) said human interface apparatus.

11. The valve arrangement of claim 10 wherein said human interface apparatus is selected from the group including:
(A) a nasal mask;
(B) an oral mask; and
(C) a nasal-oral mask.

12. The valve arrangement of claim 11 wherein said actuating element is further configured to power-down the compressor when actuated.

13. The valve arrangement of claim 9 further comprising:
(c) a microphone for detecting speech of the user, wherein, said actuator is operative to move said shutter to said second position in response to detection of said speech by said microphone.

14. The valve arrangement of claim 1 wherein, when said shutter is in said second position, the valve is configured to allow free communication of ambient air between the valve and the user.

15. A valve arrangement, operative to be interposed between a compressor and a user, in a respiratory pressure support system, comprising:
(a) a connecting element for operatively connecting a breathing tube and a human interface apparatus; and
(b) a shutter formed within said connecting element, said shutter being operative to be placed alternatively in a first position for allowing positive pressurized air flow from the compressor to the user and in a second position for preventing positive pressurized air flow from the compressor to the user, wherein said shutter is reversibly movable between said first position and said second position.

16. The valve arrangement of claim 15 wherein said connecting element includes conical shaped connecting edges for reversibly operatively connecting said connecting element to the compressor and to said human interface apparatus.

17. The valve arrangement of claim 16 wherein said connecting element is a connector having a diameter ranging in length between 8 mm and 35 mm.

18. The valve arrangement of claim 15 wherein said shutter in said first position defines an air vent between said shutter and said connecting element.

19. The valve arrangement of claim 15 wherein said human interface apparatus is selected from the group including:
(i) a nasal mask;
(ii) an oral mask; and
(iii) a nasal-oral mask.
